(19) United States
(12) Patent Application Publication
(10) Pub. No.: US 2005/0263150 A1
(43) Pub. Date: Dec. 1, 2005

(54) Systems and methods for the administration of drugs and medications

(76) Inventors: Yashwant Gilbert Chathampally, New York, NY (US); Brian Grant Klokeid, Seattle, WA (US)

Correspondence Address: SPECKMAN LAW GROUP PLLC, 1501 WESTERN AVE, SEATTLE, WA, 98101, US

(21) Appl. No.: 11/142,083

(22) Filed: May 31, 2005

Related U.S. Application Data
(60) Provisional application No. 60/575,505, filed on Jun. 1, 2004.

(51) Int. Cl. A61M 15/00; A61M 11/00

(52) U.S. Cl. 128/200.14; 128/200.21; 128/206.12; 128/203.12; 128/203.16

(57) ABSTRACT

Disclosed herein are systems and methods for the administration of drugs and/or medications to a patient. Systems and methods of the present invention are useful for achieving the non-invasive administration of drugs and/or medications, typically via a nebulization chamber in combination with an airtight face mask. Within certain embodiments, the present invention further employs a filtration device to scavenge drugs and/or medications that would otherwise escape into the patient's immediate surroundings.

### CROSS REFERENCE TO RELATED APPLICATIONS

This application claims priority under 35 U.S.C. 119(e) to U.S. Provisional Patent Application No. 60/575,505, filed Jun. 1, 2004.

### BACKGROUND OF THE INVENTION

1. Technical Field of the Invention

The present invention relates generally to systems and methods for the non-invasive administration of drugs and/or medications to a patient. Systems and methods disclosed herein advantageously employ a nebulization chamber in combination with an airtight face mask, and optionally in further combination with a filtration unit, to achieve the non-invasive administration of drugs and/or medications and the simultaneous scavenging of exhaled drugs and/or medications that would otherwise escape into the patient's immediate surroundings.

2. Background of the Invention

In medical settings, particularly in emergency department and other ambulatory procedure settings, including military field medical management settings, administration of drugs and medications having anesthetic properties, such as ketamine, is regularly necessary, particularly in pediatric populations. Generally, methods of administering anesthetic medications include intramuscular injection and/or venipuncture. For example, anesthesia can be achieved within minutes by the intramuscular injection of ketamine, and the injection is followed by the acquisition of intravenous access and appropriate monitoring. These methods of administration of anesthetic medications can, however, produce additional anxiety in patients, thus contributing to their level of discomfort. Alternatively, in some medically urgent or emergent settings, i.e. military filed medical management, it may be difficult or impossible to quickly acquire venous access.

In order for providers of anesthetic medications to achieve rapid and painless sedation among patients, without compromising patients' anxiety and comfort and without hindering subsequent intravenous access and monitoring, inhalation of nebulized anesthetic medications can be used as a method of administrating medications. Additionally, inhalation of nebulized anesthetic medications would provide an easier administration route for medical staff, since patient aversion to injections is avoided. One difficulty with such administration that is immediately evident is that inhalation of aerosolized or nebulized medications is followed naturally by exhalation, and as not all medication is absorbed by the patient, the result is the exhalation of potentially potent medication(s) into the environment. Unintended and prolonged inhalation and absorption of many anesthetic medications, such as ketamine, from the environment immediately surrounding the patient can produce adverse effects on individuals, particularly medical staff and family members who are in the patient's proximity.

Laanen, U.S. Pat. No. 4,865,027 discloses a reservoir bag connected in series to both a drug nebulizer and a non-rebreathing mask having an inlet with a one-way valve. The Laanen patent discloses a therapeutic respiratory apparatus used to provide a continuous dosage of an aerosolized medication to a patient. The apparatus has a nebulizer, a mask and a collapsible chamber. Oxygen may serve as a carrier gas supplied to the nebulizer which contains a reservoir of the liquid medication. The aerosolized medication is then delivered to a collapsible chamber which serves to store the aerosol between inhalations by a patient.

Hoppough, U.S. Pat. No. 4,886,055 discloses a nebulizer having a face mask. The '055 patent discloses an arrangement wherein humidified gas is supplied directly into a mask from a nebulizer device. The nebulizer device includes a fluid reservoir having a capillary tube. A duct sends oxygen past the upper opening of the tube so as to induce the drawing of fluid through the tube from the reservoir. The fluid is thus entrained in the oxygen supplied to a patient.

Hilliard, U.S. Pat. No. 5,586,551 discloses a non-rebreather oxygen mask in combination with a nebulizer unit wherein oxygen and an aerosolized medication are separately delivered to the mask through a one-way valve. Riggs, U.S. Pat. No. 5,277,175 discloses a nebulizer having a face mask. Vidal, U.S. Pat. No. 3,977,432 discloses an oxygen mask having an oxygen diluting device. Camp, U.S. Pat. No. 3,894,537 discloses a nebulizer having a face mask. Esbenshade, U.S. Pat. No. 3,769,973 discloses a nebulizer in series with an oxygen supply to a mouth piece. Barnes, U.S. Pat. No. 3,667,463 discloses a mask for supplying anesthetic mixed in with an oxygen stream to a patient. Schroder, U.S. Pat. No. 1,693,730 discloses a mask for supplying anesthetic mixed in with an oxygen stream to a patient.

Also disclosed in the art are respiratory inhaling devices that have at least one inlet port and employ a means such as a mask or mouthpiece to connect the device to a patient. Representative examples of such respiratory inhaling devices are disclosed in Suprenant, U.S. Pat. No. 3,666,955; Sarnoff, U.S. Pat. No. 4,433,684; Bordoni, U.S. Pat. No. 4,598,704; Jackson, U.S. Pat. No. 4,829,998; Fry, U.S. Pat. No. 4,938,209; Brown, U.S. Pat. No. 5,018,519; Michaels, U.S. Pat. No. 5,099,833; and Roberts, U.S. Pat. No. 5,119,807.

Of these examples, however, only the Laanen '027 and Hoppough '055 patents disclose the administration of oxygen and an atomized therapeutic medication. None of the aforementioned patents disclose the combination of a nebulizer unit and a face mask in further combination with a filtration unit for the administration of a drug and/or medication and the simultaneous scavenging of exhaled and/or un-administered nebulized drug and/or medication.

### SUMMARY OF THE INVENTION

Thus, it is an object of the present invention to provide systems and methods for the administration to a patient in need thereof of medications wherein the systems and methods minimize the leakage of potentially harmful medications and/or drugs into the environment immediately surrounding the patient. Within certain embodiments, inventive systems disclosed herein employ a nebulization unit and a face mask. Within other embodiments, such systems further comprise a filtration unit to scavenge excess medication and/or drugs that would otherwise escape into the patient's immediate environment.

Nebulization units disclosed herein are generally sealably and insertably connected to a face mask. Typically, a tubular member connects one end of a nebulization unit to the face mask at a one-way valve that prevents flow of drugs and/or medications traveling to the face mask back into the nebulization unit.

A filtration unit may be connected to the face mask by an outgoing tubular member. A first end of the outgoing tubular member typically connects to the filtration unit and a second end of the outgoing tubular member connects to the face mask at an outgoing one-way valve that is insertably connected to the inner frame of the face mask and directs the flow of exhaled and/or unused drug and/or medication into the filtration unit.

### BRIEF DESCRIPTION OF THE FIGURES

The present invention will be described in greater detail in the following detailed description, with reference to the accompanying drawing, wherein:

FIG. 1 discloses a perspective view of one embodiment of an inventive system for the administration and delivery of medications and/or drugs.

### DETAILED DESCRIPTION OF THE INVENTION

As indicated above, the present invention is directed to systems and methods for the non-invasive pulmonary delivery of drugs and/or medications, including, for example, anesthetics and sedatives, such as ketamine. Presented herein are systems and methods that employ a nebulization unit in combination with a face mask to achieve the rapid, painless administration to a patient in need thereof of drugs and/or medications. Within certain embodiments, systems and methods of the present invention further comprise a filtration unit to scavenge unused drugs and/or medications thereby preventing their release into the patient's immediate environment. The invention disclosed herein is described, in part, by reference to various journal articles, books, and patents. Each reference presented herein is incorporated by reference in its entirety.

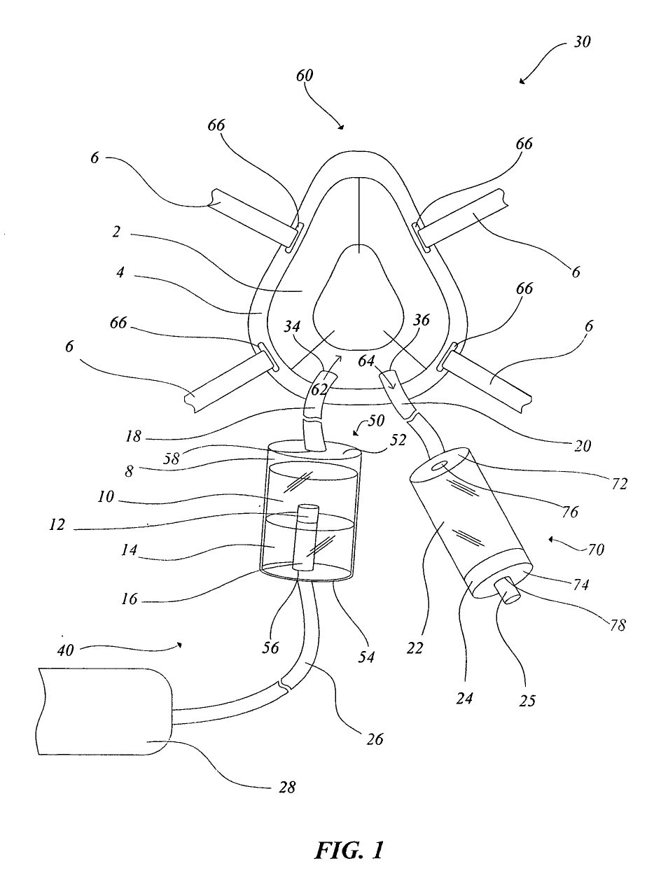

FIG. 1 presents one embodiment of an inventive system 30 that is provided with a nebulization unit 40, a face mask 60, and a filtration unit 70. Nebulization unit 40 is sealably and insertably connected to face mask 60 by an incoming tubular member 18. First end of incoming tubular member 18 connects to nebulization unit 40 and second end of incoming tubular member 18 connects to face mask 60 at an incoming one-way valve 34 that is insertably connected to an inner frame 2 of face mask 60. Filtration unit 70 is sealably and insertably connected to face mask 60 by an outgoing tubular member 20. First end of outgoing tubular member 20 connects to filtration unit 70 and second end of outgoing tubular member 20 connects to face mask 60 at an outgoing one-way valve 36 that is insertably connected to inner frame 2 of face mask 60. It will be understood that the present invention also contemplates direct connections between nebulization unit 40 and face mask 60 and/or face mask 60 and filtration unit 70 without use of either tubular member 18 and/or 20, at incoming and outgoing one-way valves 34 and 36, respectively.

Nebulization unit 40 is provided with an oxygen source 28 that is sealably and insertably connected to a nebulization chamber 50 via a connecting tubular member 26. First end of connecting tubular member 26 connects to oxygen source 28 and second end of connecting tubular member 26 connects to nebulization chamber 50. Oxygen source 28 may be provided in the form of, but is not limited to, bottled pressurized oxygen, liquefied oxygen, oxygen tanks, and the like, whereby the oxygen source 28 is supplied to nebulization chamber 50 via connecting tubular member 26.

Nebulization chamber 50 may be constructed in a variety of different sizes and configurations, as described herein below, and is provided with a body 8 and at least two ends 52 and 54. Ends 52 and 54 are each provided with an aperture 56 and 58 positioned generally at a center of ends 52 and 54. Second end of connecting tubular member 26 is insertably and sealably connected to aperture 56 of end 54, while first end of incoming tubular member 18 is insertably and sealably connected to aperture 58 of end 52. An oxygen port 12 is integrated with end 54 generally at a center of interior of end 54. Oxygen port 12 is provided with a base 16 and a bottom of base 16 is aligned with aperture 56 of end 54 so that oxygen port 12 receives and sealably engages connecting tubular member 26. Oxygen port 12 allows for high-flow oxygen from oxygen source 28 to flow towards nebulization chamber 50.

Medications and/or drugs 14 to be nebulized and delivered to face mask 60 are contained within nebulization chamber 50. Nebulization chamber 50 has the capacity to hold at least one unit dose of medication 14 to achieve an appropriate level of bioavailability. Nebulized medication 14 is introduced from nebulization chamber 50; travels through incoming tubular member 18, and enters into face mask 2 from incoming one-way valve 34.

Face mask 60 is provided with at least two apertures at its inner frame 2. Incoming one-way valve 34 is insertably and sealably connected to one of the at least two apertures and outgoing one-way valve 36 is insertably and sealably connected to the other one of the at least two apertures. Incoming one-way valve 34 allows only for a patient's one way inhalation of the nebulized medication 14 produced from nebulization chamber 50 into face mask 60, as indicated by directional arrow 62 in FIG. 1. Outgoing one-way valve 36 allows only for the patient's one way exhalation from face mask 60 towards the direction of adsorption chamber 70, as indicated by directional arrow 64 in FIG. 1.

Face mask 60 is further provided with a plurality of connecting members 66, positioned about its perimeter seal 4, to which a plurality of elastic members 6 may be releasably and adjustedly attached. Generally, the plurality of elastic members 6, which may be adjusted according to the size of a patient's head, couple with perimeter seal 4 of face mask 60 provides a tight seal when face mask 60 is placed on a patient's face so that his or her nose and mouth are enclosed by face mask 60. The tight seal substantially eliminates leakage of the nebulized medication 14 or the patient's exhaled air into the local and surrounding environment. Alternatively, face mask 60 may be provided with a port to which a connection may be attached to provide continuous suction to achieve direct elimination of exhaled and/or unused nebulized drug and/or medication.

In the present embodiment shown in FIG. 1, filtration unit 70 is sealably and insertably connected to outgoing one-way valve 36 of face mask 60 via outgoing tubular member 20. First end of outgoing tubular member 20 connects to filtration unit 70 and second end of outgoing tubular member 20 connects to face mask 60 at outgoing one-way valve 36. Filtration unit 70 may be constructed in a variety of different sizes and configurations, as described herein below, and is typically provided with a body 22 and at least two ends 72 and 74. Ends 72 and 74 are each provided with apertures 76 and 78 that are positioned generally at a center of ends 72 and 74. First end of outgoing tubular member 20 is insertably and sealably connected to aperture 76 of end 74.

Filtration unit 70 is provided with at least one filtration material, the selection of which is may be achieved through routine experimentation by those of skill in the art in view of the guidance provided herein, below. The at least one filtration material scavenges any medication and/or drug remaining in a patient's exhaled air. Suitable exemplary filtration materials are described in further detail herein, below, and include, but are not limited to, activated charcoal, sphagnum moss, porous and/or affinity media, and the like.

Filtration unit 70 may be further provided with an indicator 25 that is fixed or removably attached to proximal end 74 of the filtration unit 70. Proximal end 74 serves an exit point for the patient's exhaled air. Indicator 25 allows for rapid confirmation that no excess medication and/or drug 14 is leaking or has leaked through inventive system 30 into the surrounding local environment. Indicator 25 is exemplified in FIG. 1 by an indicator strip, such as a pH (Litmus) paper, that changes color upon detection of the patient's exhaled air. Other suitable indicators are described in greater detail herein below.

The inventive system described herein is suitable for use with drugs and/or medications including, but are not limited to, ketamine, diazepam, lorazepam, midazolam, and the like, that cannot be safely be exhaled/disseminated directly into the patient's local environment. Other drugs and/or medications that may be suitably administered by the systems and methods presented herein are described in greater detail herein below.

Aerosolizers and Aerosolization Chambers Including Nebulizers and Nebulization Chambers

As indicated in the exemplary embodiment presented herein above, and in FIG. 1, the systems and methods of the present invention utilize one or more aerosolizer(s), aerosolization chamber(s) nebulizer(s), nebulization chamber(s), atomizer(s), and/or atomizer chanber(s) to produce a mist of drug- and/or medication-containinig droplets, typically water or saline droplets, for inhalation. These drug-containing droplets are referred to herein as inhaled pharmaceutical aerosols (IPAs). When the lung is the target for the aerosol, the inhaled aerosol ideally comprises particles within a certain size range. As used herein, the terms aerosolizer(s), aerosolization chamber(s) nebulizer(s), nebulization chamber(s), atomizer(s), and/or atomizer chamber(s) are used interchangeably to refer a device for producing a mist of drug- and/or medication-containing droplets that are suited for pulmonary administration. Typically, IPAs suitable for pulmonary administration of drugs and/or medications have diameters in the range of about 0.1 μm to about 50 μm, more typically between about 0.5 μm and about 10 μm. It will be understood, however, that the speed of the inhaled air plays a significant role in determining what size of particles will deposit within the respiratory tract. Droplet evaporation and/or condensation will differ with aerosol composition and, consequently, will yield different deposition patterns. See, for example, Finlay, “The Mechanics of Inhaled Pharmaceutical Aerosols: An Introduction” (Academic Press, 2001) and Hickey, “Inhalation Aerosols” (ed. A. J, Marcel Dekker, N.Y., 1996), incorporated herein by reference in its entirety.

Nebulizers that may be suitably employed with the systems and methods disclosed herein are typically classified into two types: ultrasonic nebulizers and jet (pneumatic) nebulizers. Jet nebulizers operate based upon the venturi principle and are more common due to their lower cost, small volume, and use a source of pressurized air, oxygen, or other gas to blast a stream of air through a drug-/medication-containing reservoir thereby producing droplets comprising the drug and/or medication. In contrast, ultrasonic nebulizers produce droplets by mechanical vibration of a plate or mesh. In either type of nebulizer, the drug is usually contained in solution in the solvent in the nebulizer and so the droplets being produced contain the drug in solution. Within certain applications, however, the drug may be contained within small particles suspended in the water, which are then contained as particles suspended inside the droplets being produced.

Important variables for both types of nebulizer are treatment time required, particle size produced, and aerosol drug output. There are several advantages to jet nebulization, including that effective use requires only simple, tidal breathing, and that dose modification and dose compounding are possible. Disadvantages include the length of treatment time and equipment size.

A wide variety of jet nebulizers available in the art may be suitably employed in the systems and methods disclosed herein. Exemplary jet nebulizers that are available from commercial sources include the following: Acorn-I and Acorn-II (Marquest Medical Products); Airlife™ Brand Misty Max 10™ (Cat. No. 002438; Cardinal Health; McGaw Park, Ill.); AquaTower; AVA-NEB; Cirrhus, Dart; DeVilbiss 646; Downdraft; Fan Jet; MB-5; Misty Neb; PARI LC JET; PARI-JET; Salter 8900; Sidestream; Updraft-II; and Whisper Jet. See, also, Wallace, U.S. Pat. No. 5,036,840 “Nebulizer System”. Design modifications to the constant-output nebulizer have resulted in breath-enhanced, open-vent nebulizers such as the Pari LC Plus and the dosimetric AeroEclipse.

In use, jet nebulizers typically contain an aqueous volume of between about 0.5 ml and about 5.0 ml, more typically between about 1 ml and about 2.5 ml. They are most frequently powered with a compressor such as, for example, the PulmoAide (DeVilbiss). Parameters considered in selecting a suitable jet nebulizer include the total output (TO), time for total output (TTO), and percent output in respirable range (PORR). TO is obtained by weighing before nebulization; TTO is calculated from initiation of nebulization; and PORR may be measured by a laser particle analyzer in continuous nebulization to the point of abrupt drop in output. The respirable particle delivery rate (RPDR) is calculated by dividing TO by TTO and multiplying by PORR. Typical RPDR are between about 0.01 ml/min to 0.5 ml/min, more typically between about 0.02 and about 0.20 ml/min. The output characteristics of commercial nebulizers vary substantially and, as a consequence, the choice of nebulizer will impact the time required for treatment as well as the total amount of drug and/or medication delivered to the lungs. See, Loffert et al., Chest 106:1788-1792 (1994).

Ultrasonic nebulizers use the converse piezoelectric effect to convert alternating current to high-frequency acoustic energy. Ultrasonic nebulizers generally have a higher output rate than jet nebulizers, but a larger average particle size. Ultrasonic nebulizers can also substantially increase reservoir solution temperature, the opposite of jet nebulizer cooling. Drug concentration in the reservoir does not increase with ultrasonic nebulization, as it does with jet nebulization. Ultrasonic nebulizers are more expensive and fragile than jet nebulizers, may cause drug degradation, and do not nebulize suspensions as well as jet nebulizers. See, Rau, Respir. Care 47(11):1257-1275 (2002).

The following ultrasonic nebulizers exemplify those that are available in the art from commercial sources: ShinMed Models 988 and 966 by Shining World Health Care (Taipei, Taiwan); NE-C21 and NE-C25 by Omron; Model 6610 by Lumiscope; Mist II Model Number 40-270-000 by MABIS; and UM20-1.6 by Hielscher.

Face Mask and Filtration Unit

Because of the potential risks and debilitating effects to medical care professionals and other individuals in the patient's immediate environment, drugs and/or medications used in combination with the systems and methods of the present invention require the use of a tightly sealing face mask to minimize the leakage of the drug and/or medication into the surrounding environment. Typically, face masks that are suitably employed in combination with the systems and methods of the present invention use a system of one-way valves that permit the inhalation of the nebulized drug and/or medication, but prevent its distribution into the surrounding environment. Thus, a one-way valve between the nebulizer and/or nebulization chamber permits the unidirectional flow of nebulized drug and/or medication into the face mask, but prevents exhaled air, including exhaled air containing excess drug and/or medication, from reentering the nebulizer and/or nebulization chamber. In those embodiments including a filtration unit, a second one-way valve is, typically, positioned between the face mask and filtration unit to permit the unidirectional flow of exhaled air and nebulized drug and/or medication through the filtration unit, but preventing air from passing from the external environment, through the filtration unit and into the face mask. Typically, face masks and filtrations units described herein are disposable, one-use, units. Depending upon the precise application contemplated, face masks may be adaptably configured to accept a variety of filtration units suitable for scavenging the drug and/or medication to be administered.

Suitable face masks that may be used in the systems and methods presented herein are readily available in the art and are exemplified by the “Pocket Mask™” by Laerdal Medical Corporation (Wappingers Falls, N.Y.) and Vital Signs self sealing mask (Totowa, N.J.). Other suitable face masks that may be suitably modified for use in conjunction with the systems and methods of the present invention are disclosed in Fry, U.S. Pat. No. 4,938,209 “Mask for a Nebulizer”; Brown, U.S. Pat. No. 5,018,519 “Mask for Administering an Anesthetic Gas to a Patient”; Smaldone, U.S. Pat. No. 6,748,949 “Face Masks for use in Pressurized Drug Delivery Systems”; Hilliard, U.S. Pat. No. 5,586,551 “Oxygen Mask with Nebulizer”; and Denyer, U.S. Pat. No. 6,192,876 “Inhalation Apparatus and Method”. Each of these patents is hereby incorporated by reference in its entirety.

Face masks suitable for use with the systems and methods presented herein are designed to provide a tight, hermetic fit between the patient's face and the mask. Within those embodiments wherein distribution and/or leakage of the nebulized drug and/or medication into the patient's immediate environment is to be minimized and/or prevented, the tightly fitting face mask will be fitted with a removable or permanently-attached filtration unit, such as a filtration cartridge. In such embodiments, the face mask also possesses a one-way exhalation valve to permit the unidirectional flow of exhaled air and unused nebulized drug and/or medication.

Exemplary masks having one-way exhalation valves are disclosed in, for example, Burns, U.S. Pat. No. 5,062,421; Japuntich, U.S. Pat. Nos. 4,827,924, 5,509,436, and 5,325,892; Vicenzi, U.S. Pat. No. 4,537,189; Braun, U.S. Pat. No. 4,934,362; and Scholey, U.S. Pat. No. 5,505,197. Commercially available products include the 5000 and 6000 Series™. Masks sold by 3M Company (St. Paul, Minn.). Optionally, an exhalation valve may be protected by a valve cover—see, for example, U.S. Design Pat. Nos. 347,299 and 347,298 that protect the valve from physical damage caused, for example, by inadvertent impacts.

Each of the exemplary prior art masks having exhalation valves are designed to prevent the wearer from directly inhaling harmful particles, but fall short of disclosing a means to protect other persons in the vicinity from being exposed to drugs and/or medications expelled by the wearer. In order to overcome this deficiency in the art, certain embodiments of the systems and methods of the present invention further provide a filtration unit to retain expelled drugs and/or medications. The importance of filtration units has been recognized in the context of maintaining sterility in the operating room environment. See, Vesley et al., “Clinical Implications of Surgical Mask Retention Efficiencies for Viable and Total Particles,” INFECTIONS IN SURGERY, 531-536 (1983) but have not, heretofore, been taught for the prevention of the distribution of exhaled and/or nebulized drugs and/or medications.

Suitable face masks that may be advantageously modified for use in combination with the nebulization of a drug and/or medication, as disclosed herein, are available in the art. Thus, for example, commercially available products include the 1800™, 1812™, 1838™, 1860™, and 8210™ brand masks sold by the 3M Company. Other examples of masks of this kind are disclosed in Kronzer, U.S. Pat. No. 5,307,706; Dyrud, U.S. Pat. No. 4,807,619; and Berg, U.S. Pat. No. 4,536,440. These masks are relatively tightly fitting to prevent gases and liquid contaminants from both entering and exiting the interior of the mask at its perimeter; however, each of these masks lacks a nebulization chamber, an inhalation valve, and an exhalation valve to permit exhaled air to be quickly purged from the mask interior.

Exemplary suitable filtration units may employ one or more of the following: micropore filters, sphagnum moss, activated charcoal, affinity reagents, and/or electrical charge-based filters. Filtration units that may be satisfactorily employed with the methods and systems disclosed herein may have high particle filtration capacity such that nebulized solvent droplets comprising drugs and/or medications are retained within the filtration unit. Typically, filtration units are effective against both gases and vapors owing to the filter's high chemisorption and physisorption capacities.

Depending upon the nature of the drug and/or medication, a wide variety of filtering materials may be selected and employed in the systems and methods disclosed herein. Thus, within certain embodiments, an entangled web of electrically charged melt-blown microfibers (BMF) may be suitable for filtering exhaled drugs and/or medications. BMF fibers typically have an average fiber diameter of about 10 micrometers (μm) or less, which is consistent with the typical range of particulate diameters yielding from conventional nebulization chambers. When randomly entangled into a web, BMF fibers have sufficient integrity to be handled as a mat.

Examples of suitable fibrous materials that may be used within filtration units of the present invention are disclosed within Baumann, U.S. Pat. No. 5,706,804; Peterson, U.S. Pat. No. 4,419,993; Mayhew U.S. Reissue Pat. No. Re 28,102; Jones U.S. Pat. Nos. 5,472,481 and 5,411,576; and Rousseau U.S. Pat. No. 5,908,598. The fibrous materials may contain additives to enhance filtration performance, such as the additives described in Crater, U.S. Pat. Nos. 5,025,052 and 5,099,026 and may also have low levels of extractable hydrocarbons to improve performance such as disclosed within Rousseau U.S. Pat. application Ser. No. 08/941,945. Fibrous webs also may be fabricated to have increased oily mist resistance as shown in Reed, U.S. Pat. No. 4,874,399 and in Rousseau, U.S. patent application Ser. Nos. 08/941,270 and 08/941,864. Electric charge can be imparted to nonwoven BMF fibrous webs using techniques described in, for example, Angadjivand, U.S. Pat. No. 5,496,507; Kubik, 4,215,682; and Nakao, U.S. Pat. No. 4,592,815. Each of the aforementioned patents is incorporated by reference herein in its entirety.

Alternative embodiments employ filtration units that comprise a spun-bonded nonwoven fibrous media. An exemplary exhale filtration unit comprises a polypropylene spunbonded web. Such a web may be obtained from PolyBond Inc. (Waynesboro, Va.; Product No. 87244). The exhale filtration unit may also employ an open-cell foam. Additionally or alternatively, if the mask uses shaping layers to provide support for the filter media, such as is disclosed in Kronzer, U.S. Pat. No. 5,307,796; Dynid, U.S. Pat. No. 4.807.619; and Berg, U.S. Pat. No. 4.536.440), the shaping layers may be used as an exhale filtration unit. Or the filtration unit may be made from the same materials that are commonly used to form shaping layers. Such materials typically include fibers that have bonding components that allow the fibers to be bonded to one another at points of fiber intersection. Such thermally bonding fibers typically come in monofilament or bicomponent form. The nonwoven fibrous construction of the shaping layer provides it with a filtering capacity that permits the shaping layer to screen out larger particles such as saliva. Because these fibrous webs are made from thermally bonding fibers, it is also possible to mold the webs into a three-dimensional configuration fashioned to fit over an exhalation valve as, for example, in the form of a valve cover. Generally, any porous structure that is capable of filtering contaminants is contemplated for use as an exhale filtration unit of the present invention. Each of the aforementioned patents is incorporated by reference herein in its entirety.

Within alternative and/or additional embodiments of the present invention, exhale filtration units may advantageously contain a fluorochemical additive(s). Fluorochemical additives are described in Crater U.S. Pat. Nos. 5,025,052 and 5,099,026; Baumann, U.S. Pat. No. 5,706,804; and Klun, U.S. Pat. No. 6,127,485. The fluorochemical additive may be incorporated into the volume of solid material that is present in the porous structure of the exhale filtration unit and/or it may be applied to the surface of the porous structure. When the porous structure is fibrous, the fluorochemical additive preferably is incorporated at least into some or all of the fibers in the exhale filter element. Each of the aforementioned patents is incorporated by reference herein in its entirety.

The fluorochemical additive(s) that may be used in connection with the exhale filtration unit to inhibit liquid passage through the element include, but are not limited to, fluorochemical oxazolidinones, fluorochemical piperazines, fluoroaliphatic radical-containing compounds, fluorochemical esters, and combinations thereof. Preferred fluorochemical additives include the fluorochemical oxazolidinones such as C₈ F₁₇ SO₂ N(CH_{3)CH2} CH(CH₂Cl)OH and fluorochemical dimer acid esters. An exemplary commercially available fluorochemical additive is FX-1801 Scotchban™ brand protector from 3M Company (Saint Paul, Minn.).

In addition to, or in lieu of, the above listed fluorochemical additives, other materials may be employed to inhibit liquid penetration such as waxes or silicones. Essentially any product that may inhibit liquid penetration but not at the expense of significantly increasing pressure drop through the exhale filtration unit is contemplated for use in this invention. Within certain embodiments, discussed above, the fluorochemical additive is melt-processable such that it is incorporated directly into the porous structure of the exhale filtration unit. The additives may desirably impart repellency to aqueous fluids and thus increase oleophobicity and hydrophobicity or are surface energy reducing agents.

Exhale filtration units for removing nebulized and/or exhaled drugs and/or medications from the filter mask ideally have suitable sorptive qualities for removing such contaminants. The filtration unit may, for example, be made from an active particulate such as activated carbon bonded together by polymeric particulate to form a filtration unit that, optionally, includes a nonwoven particulate filter as described above to provide drug/medication removal characteristics alone and/or in combination with satisfactory particulate filtering capability. Exemplary bonded particulate filters are disclosed in Braun, U.S. Pat. Nos. 5,656,368, 5,078,132, and 5,033,465; and Senkus, U.S. Pat. No. 5,696,199. An exemplary filtration element having combined gaseous and particulate filtering abilities is disclosed in Braun, U.S. Pat. No. 5,763,078. Filtration units may also be configured as a nonwoven web of, for example, melt-blown microfibers that further comprises an active particulate such as described in Braun, U.S. Pat. No. 3,971,373. The active particulate also can be treated with topical treatments to provide vapor removal. See, e.g., Abler, U.S. Pat. Nos. 5,496,785 and 5,344,626. Each of the aforementioned patents is incorporated by reference herein in its entirety.

Within further embodiments, filtration units employed in the systems and methods disclosed herein may utilize a porous membrane that is capable of imbibing a liquid. Typically, porous membranes have pore sizes suitable for trapping nebulized water particles wherein the water particles are typically about 10 nm to about 100 μm, more typically between about 0.1 μm to about 10 μm. Membrane thicknesses are generally between about 2.5 μm and about 2500 μm, or between about 25 μm and about 250 μm.

Porous membranes fabricated out of a wide variety of materials are contemplated, the choice of which will largely depend upon the precise drug and/or medication being administered as well as its solvent composition. For example, porous membranes may be prepared of polytetrafluoroethylene or thermoplastic polymers such as polyolefins, polyamides, polyimides, polyesters, and the like. Examples of suitable membranes include, for example, those disclosed in Shipman, U.S. Pat. No. 4,539,256; Mrozinski, U.S. Pat. No. 4,726,989; and Gore, U.S. Pat. No. 3,953,566, each of which patents are hereby incorporated by reference.

An exemplary, suitable, commercially available porous membrane is the microporous polypropylene membrane material having the brand name CELGARD™2400 (Hoechst Celanese Corp.) having a thickness of 0.0024 cm. Depending upon the precise application contemplated, such a membrane may be imbibed, alternatively, with a heavy white mineral oil (available from Aldrich Chemical Co.); polypropylene glycol diol (e.g., 625 molecular weight, available from Aldrich Chemical Co.); heavy white mineral oil in xylene (boiling range 137°-144° C., available from EM Science) at concentrations of, for example, 5, 10, 15, 20, and 25 percent by volume.

Alternatively, a filtration unit may employ a molecular sieve material such as, for example, 14-30 Mesh (U.S. Sieves Standard), preferably having a nominal pore size of 4 Angstroms with moisture content of less than 1.5% wt. A material presently commercially available is catalogued as Grade 516 by Davidson Chemical Division, W. R. Grace and Company (Baltimore, Md.). Sieve material in powdered form of approximately 4 Angstrom pore size may also be used.

Sieve material, in functioning to selectively adsorb molecules in the 4 Angstrom size (e.g., H₂O) over larger molecules, “dries” nebulized particulates entering the filtration unit thereby affording immediate intimate contact between the water molecules and, optionally, an oxidizing color change indicator material (see below).

Face masks having exhale filtration units according to the present invention meet or exceed industry standards for characteristics such as fluid resistance, filter efficiency, and wearer comfort. In the medical field, the bacterial filter efficiency (BFE), which is the ability of a mask to remove particles, usually bacteria expelled by the wearer, is typically evaluated for face masks. BFE tests are designed to evaluate the percentage of particles that escape from the mask interior. There are three tests specified by the Department of Defense and published under MIL-M-36954C, Military Specification: Mask, Surgical, Disposable (Jun. 12, 1975), which evaluate BFE. As a minimum industry standard, a surgical product should have an efficiency of at least 95% when evaluated under these tests.

BFE is calculated by subtracting the percent penetration from 100%. The percent penetration is the ratio of the number of particles downstream to the mask to the number of particles upstream to the mask. Filtering face masks that use a polypropylene BMF electrically-charged web and have an exhale filter element according to the present invention are able to exceed the minimum industry standard and may even have an efficiency greater than 97%.

Indicators of the End of a Filtration Unit's Useful Life

In order to signal to the user the end of a filtration unit's useful life, and to prevent the unwanted escape of drug and/or medication into the surrounding environment, the present invention further contemplates the use of filter units that further comprise one or more indicator. Thus, systems and methods of the present invention may comprise one or more indicator to indicate the remaining time of use by, for example, optical, auditory, and/or electrical signs to warn the user in time of the imminent exhaustion of the filter material.

Indicator strips may change color and/or possess some other indicator property that indicates that the drug is not being effectively blocked by the filter and/or is passing through the filter and into the environment surrounding the patient. This indicates that the filter's useful life is expired and that a new filter is required. Within certain embodiments, indicator strips are drug-specific and contain a chemical that reacts with the drug being administered. Alternatively, when used in combination with filtration units that operate by physically trapping particulate matter, such as for example, water droplets, indicator strips may detect increases in moisture and/or humidity in the filtration unit.

Exemplary suitable indicators include the Easy Cap II CO₂ and the End-tidal single use detectors by Nellcor Purital Bettett™, Inc. (Pleasanton, Calif.; U.S. Pat. Nos. 4,728,499; 4,879,999; 5,166,075; and 5,179,002) as well as those presented in Jones, U.S. Pat. No. 4,530,706 “Respirator Cartridge End-of-service Life Indicator” and references disclosed therein, each of which is incorporated herein by reference. For example, U.S. Pat. No. 4,154,586 discloses windowed color changing indicators that provide desirable means for monitoring the effectiveness of organic vapor respirator cartridges.

More specifically, the Jones, '706 patent discloses upstream molecular sieving of atmospheres directed through color indicating chemicals of respirator cartridge units. Moisture molecules are removed from vapors to be monitored so that the “dried” vapors make immediate contact with a color indicating (oxidizing) chemical. Indicators may be contained within a capsule and positioned within the filtration unit for viewing through the wall of the filtration unit. Other forms of shell windowing may also be suitable.

Regardless of the precise configuration of the indicator within the filtration unit, a wide variety of indicator agents may be employed. For example, a dried solution of a reagent grade sodium dichromate in sulfuric acid and water may be supported by granular silica gel as described in U.S. Pat. No. 4,154,586. It should be understood, however, that the present invention is not intended to be limited to specific details of indicator composition. A suitable silica gel is Grade 408, 12-29 Mesh (Tyler Sieve) having a density of approximately 47 lbs. per ft.³ (Davidson Chemical Division, W. R. Grace and Company, Baltimore, Md.).

Alternative passive and active indicator systems for use with the filtration units disclosed herein are described in Debe, U.S. Pat. No. 5,659,296 “Exposure indicating apparatus.” Passive indicators typically include a chemically coated paper strip(s) that changes color when the sorbent material is near depletion. Passive indicators require active monitoring by the user. Active indicators include a sensor that monitors the level of drug and/or medication passing through a filtration unit and typically provides an automatic warning to the user.

One type of active indicator is an exposure monitor, which is a relatively high cost device that may monitor concentrations of escaping moisture and/or solvent and/or one or more drug and/or medication. Such active indicator systems typically detect when a threshold limit value is exceeded.

A second type of active indicator uses a sensor either embedded in the sorbent material or in the air stream downstream of the filtration unit and connected to an audible or visual signaling device. The filtration unit containing the sorbent material is replaced when the sensor detects the presence of a predetermined concentration of a target molecule.

Some exposure indicators include threshold devices that actuate a visual or audible alarm when a certain threshold level or levels have been reached. In addition, some active indicators also provide a test function for indicating that the active indicator is in a state of readiness, e.g., the batteries of the indicator are properly functioning. Ideally, indicators provide an indication of the rate of change of a target molecule above the threshold level and/or sense the remaining useful life of the filtration unit. For some applications, it is useful to identify decreasing concentrations of a target molecule, such as oxygen.

By sampling air after it has passed through the sorbent material of the filtration unit, or at some intermediate location within the filtration unit, a reversible sensor can detect the end-of-life of the filtration unit. A processing device for generating a concentration signal responsive to at least one property of the reversible sensor may be releasably attached to the filtration unit so that it can be removed without interrupting the flow of air through the filtration unit. The processing device provides an active indication, such as audio, visual, or tactile response to the concentration signal. The sensor may be coupled to the processing device by an optical, electrical, or general electromagnetic coupler covering the frequency range, for example, from DC to RF to microwave.

Drugs and/or Medications Suitable for Administration

As indicated above, systems and methods of the present invention may be suitably employed for the rapid, non-invasive, pulmonary administration of a wide variety of drugs and/or medications including, but not limited to, anesthetics, sedatives, analgesics, paralytics and/or neuromuscular blockers, reversal agents, antihistamines, anxiolytics, anticholinergics, and/or antihistaminergics. Each of the drugs and/or medications that are used in combination with the systems and methods disclosed herein have in common one or more property requiring that the distribution into the environment surrounding the patient to which the drug/medication is administered should be minimized and/or avoided altogether. Drugs and/or medications that may be suitably employed in conjunction with the systems and methods of the present invention may be formulated into pharmaceutical compositions for pulmonary administration via nebulization or the like.

Drugs and/or medications disclosed herein are preferably formulated as pharmaceutical compositions appropriate for pulmonary administration. Suitable formulations are discussed in detail, herein. Such formulations are typically prepared at a pH that is optimized for solubility, drug stability, and/or absorption through the pulmonary mucosa.

A “therapeutically effective amount” of a drug is an amount sufficient to demonstrate a desired activity of the drug—that is, a dose that is effective in facilitating a desired physiological effect. The actual dose will vary, depending on the body weight of the patient, the severity of the patient's condition, the nature of medications administered, the number of doses to be administered per unit of time, and other factors generally considered by the ordinary skilled physician in the administration of drugs.

As used herein, the term “nebulization” refers to the change of a liquid medicine into fine droplets (in aerosol or mist form) that are inhaled through mask. As used herein, the term “dispersant” refers to an agent that assists nebulization of the drug and/or medicine or absorption of the drug and/or medicine into the pulmonary tissue, or both. In a specific aspect, the dispersant can be a mucosal penetration enhancer. Preferably, the dispersant is pharmaceutically acceptable. As used herein, the term “pharmaceutically acceptable” means approved by a regulatory agency of the Federal or a state government or listed in the U.S. Pharmacopeia or other generally recognized pharmacopeia for use in animals, and more particularly in humans. As used herein, the term “nebulizer” refers to a device used to deliver inhaled medications, in which an air compressor is used to blow an atomized medication through a mouthpiece or face mask. Typically, a “nebulizer” changes liquid medicine into fine droplets (in aerosol or mist form) that are inhaled through a mouthpiece or mask. A nebulizer may, advantageously, be used for babies and children too small to be able to coordinate using a metered dose inhaler (MDI).

Suitable dispersing agents suitable for use with the nebulization systems and methods disclosed herein are well known in the art, and include but are not limited to surfactants and the like. For example, surfactants that are generally used in the art to reduce surface induced aggregation of drugs and/or medicines caused by atomization of the solution forming the liquid aerosol may be used. Non-limiting examples of such surfactants are surfactants such as polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art, and can be selected on the basis of desired properties, depending on the specific formulation, concentration of drug and/or medication, or diluent (in a liquid formnulation).

Within certain embodiments, liquid aerosol formulations contain a drug and/or medication and a dispersing agent in a physiologically acceptable diluent. That is, it must be broken down into liquid or solid particles in order to ensure that the aerosolized dose actually reaches the mucous membranes of the nasal passages or the lung. The term “aerosol particle” is used herein to describe the liquid or solid particle suitable for pulmonary administration, i.e., that will reach the mucous membranes. Other considerations, such as construction of the delivery device, additional components in the formulation, and particle characteristics are important. These aspects of pulmonary administration of a drug are well known in the art, and manipulation of formulations, nebulization means and construction of a delivery device require at most routine experimentation by one of ordinary skill in the art in view of the exemplary system described herein and presented in FIG. 1. In a particular embodiment, the mass median dynamic diameter will be 5 micrometers or less in order to ensure that the drug particles reach the lung alveoli. Wearley, Crit. Rev. in Ther. Drug Carrier Systems 8:333 (1991).

The present invention provides aerosol formulations and dosage forms for use, for example, in treating patients suffering from pain and/or in need of sedation. In general such dosage forms contain a drug and/or medication in a pharmaceutically acceptable diluent including, but are not limited to, sterile water, saline, buffered saline, dextrose solution, and the like. In a specific embodiment, a diluent that may be used in the present invention or the pharmaceutical formulation of the present invention is phosphate buffered saline, or a buffered saline solution generally between the pH 7.0-8.0 range, or water.

The liquid aerosol formulation of the present invention may include, as optional ingredients, pharmaceutically acceptable carriers, diluents, solubilizing or emulsifying agents, surfactants and excipients. The formulations of the present embodiment may also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

Systems and methods of the present invention may employ and/or be used in combination with a wide variety of anesthetics, sedatives, and analgesics that are readily available in the art. As used herein, the term “anesthetics” generally refers to that class of compounds that are normally used to produce loss of consciousness before and during surgery. In addition, anesthetics may be given in small amounts, as sedatives and/or analgesics, to relieve anxiety and/or or pain, respectively, without causing unconsciousness.

Thus, as used herein, the term “anesthetics” is meant to include, without limitation, benzodiazepine, enflurane, etomidate, halothane, isoflurane, ketamine, methohexital, methoxyflurane, nitrous oxide, non-opioids, opioids, propofol, and thiopental. Opioids are frequently administered in a clinical situation wherein the patient presents with severe pain and/or in situations requiring procedural sedation. Common opioids include compounds such as morphine, meperidine, hydromorphone, oxymorphone, methadone, levorphanol, fentanyl, oxycodone, codeine, hydrocodone, hydromorphone, methadone, levorphanol, tramadol, pentazocine, nalbuphine, butorphanol, buprenorphine, and dezocine. See, for example, Blackburn and Vissers, Emergency Medicine Clinics of North America 18(4) (2000), incorporated herein by reference in its entirety.

Anesthetics are exemplified in further detail herein by ketamine ((2-(2-chlorophenyl)-2-(methylamino)-cyclohexanone). It will, however, be recognized by those skilled in the art that alternative anesthetics may be advantageously utilized depending upon the precise application contemplated. Nasal, caudal, intrarectal, subcutaneous (s.c.), intramuscular (i.m.), and intravenous (i.v.) administration of ketamine to achieve sedation are described in Louon et al., Br. I. Ophthalmol. 77:529-530 (1993); and Weksler et al., Can. J. Anaesthesia 40:119-121 (1993). Ketamine is also known to have analgesic properties when administered in subanesthetic doses. See, for example, Domino et al., Clin. Pharmacol. Ther. 6:279 (1965); Boyill, Br. I. Anaesth. 43:496 (1971); and Sadove et al., Anesth. Analg. 50:452-457 (1971).

Ketamine dosage suitable for pulmonary administration according to the systems and methods of the present invention will vary depending upon the precise application as well as the patient age and/or condition. Typically, however, ketamine may be administered pulmonarily by the systems and methods disclosed herein at approximately 0.01 mg/kg to approximately 20 mg/kg of body weight. More typically, the dose of ketamine will be approximately 0.05 mg/kg to approximately 4 mg/kg of body weight. Still more typically, the dose of ketamine will be approximately 0.2 mg/kg to approximately 1 mg/kg of body weight. The total dose of ketamine for pulmonary administration may be between approximately 1 mg to about 300 mg.

As noted above, typically, anesthetics may be administered to achieve analgesic effects. In such instances, anesthetics such as ketamine are generally administered in an amount that is about 10% to about 20% of the amount used to induce anesthesia. Preferably, the effective dose is titrated under the supervision of a physician and/or other medical care provider, so that the optimum dose for the particular application is accurately determined. Thus, the present invention provides a dose suited to each individual patient.

Ketamine may be advantageously combined with benzodiazepines and barbiturates to extend ketamine's half-life, thereby prolonging clinical recovery time by about 30%. Furthermore, addition of benzodiazepines may reduce the incidence of hallucinatory emergence reactions and may be advantageously used in combination with ketamine if risk factors are present. Such risk factors include, but are not limited to: age in excess of 10 years, rapid administration, excessive noise or stimulation or a baseline of frequent dreaming. Co-administration of ketamine and benzodiazepines is generally not recommended whereas concurrent administration of an anticholinergic may be recommended to reduce ketamine-induced hypersalivation. Atropine and/or glycopyrrolate may also be combined with ketamine.

Propofol is an ultrashort-acting sedative-hypnotic unrelated to the benzodiazepines or barbiturates. Propofol is an isopropylphenol that is typically formulated as an aqueous emulsion in soybean oil and is almost completely insoluble in water. Propofol may be advantageously administered for short-term procedural sedation. Typically, propofol is administered from approximately 0.10 mg/kg to approximately 5 mg/kg. Still more typically, propofol is administered from approximately 0.50 mg/kg to approximately 2 mg/kg.

Nitrous oxide is a safe and effective sedative/analgesic, exhibits a rapid onset of action of 3 to 5 minutes, and exhibits a duration of action of 3 to 5 minutes. Typically, nitrous oxide is administered as a 50:50 N₂O: O₂ mixture to prevent hypoxia.

Fentanyl is a synthetic opioid having approximately 1000 times the potency of meperidine. Fentanyl has an extremely rapid onset of action and a short duration of action. It is a frequent choice for analgesia and procedural sedation. Typical dosages for fentanyl are between about 0.10 μg/kg to about 20 μg/kg every 1 to 2 minutes. More typical dosages for fentanyl are between about 0.50 μg/kg to about 5 μg/kg every 1 to 2 minutes.

Methohexital (Brevital) is a barbiturate suitably employed in combination with the systems and methods disclosed herein to achieve procedural sedation and analgesia. Methohexital has an onset of action of less than 1 minute and a duration of action of less than 10 minutes making it a suitable choice for reduction of fractures or cardioversions. Because methohexital is purely an amnestic agent and has no analgesic properties, it may be advantageous to administer small doses of opioids in combination with methohexital. Typically, suitable doses of methohexital are between approximately 0.2 mg/kg to approximately 5 mg/kg. More typical does of methohexital are between approximately 0.5 mg/kg and approximately 2 mg/kg.

Systems and methods of the present invention may alternatively employ and/or be used in combination with (1) paralytics and/or neuromuscular blockers including, without limitation, succinylcholine (with or without atropine), vecuronium, rocuronium, and pancuronium; (2) reversal agents including, without limitation, naloxone; (3) antihistamines including, without limitation, phenergan; and (4) anxiolytic, anticholinergic, and/or antihistaminergic agents including, without limitation, benadryl (diphenhydramine) and hydroxyzine.

Midazolam (Versed) is a benzodiazepine having both amnestic and anxiolytic properties. Because it lacks analgesic effects, it is commonly administered in combination with fentanyl to create a cocktail suitable for procedural and/or conscious sedation—in some instances, this combination is preferred over benzodiazepine. Midazolam has a more rapid onset of action than does diazepam (Valium) and, consequently, is more suitable for use in emergency situations. Typically, midazolam is administered at a dose of between about 0.01 mg/kg to about 1.0 mg/kg, more typically between about 0.05 mg/kg and about 0.5 mg/kg. When used in combination with fentanyl, a suitable doing regimen includes about 0.5 μg of fentanyl for each 0.05 mg of midazolam.

1. A system for the non-invasive administration of a drug and/or medication to a patient, said system comprising:
(a) a face mask;
(b) a nebulizer; and
(c) a filtration unit;
wherein said nebulizer is sealably and insertably connected to said face mask at a first one-way valve and wherein said filtration unit is sealably and insertably connected to said face mask at a second one-way valve;
wherein said first one-way valve prevents the flow of said drug and/or medication from said face mask back into said nebulization unit; and
wherein said second one-way valve prevents the flow of said drug and/or medication from said filtration unit back into said face mask.

2. The system of claim 1 wherein said nebulizer is capable of producing a mist of drug- and/or medication-containing droplets having diameters in the range of about 0.1 μm to about 50 μm.

3. The system of claim 2 wherein said nebulizer is capable of producing a mist of drug- and/or medication-containing droplets having diameters in the range of about 0.5 μm to about 10 μm.

4. The system of claim 1 wherein said nebulizer is selected from the group consisting of an ultrasonic nebulizer and a jet nebulizer.

5. The system of claim 4 wherein said nebulizer is a jet nebulizer selected from the group consisting of: Acorn-I and Acorn-II (Marquest Medical Products); Airlife™ Brand Misty Max 10™ (Cat. No. 002438; Cardinal Health; McGaw Park, Ill.); AquaTower; AVA-NEB; Cirrhus, Dart; DeVilbiss 646; Downdraft; Fan Jet; MB-5; Misty Neb; PARI LC JET; PARI-JET; Salter 8900; Sidestream; Updraft-II; and Whisper Jet.

6. The system of claim 4 wherein said nebulizer in an ultrasonic nebulizer selected from the group consisting of: ShinMed Models 988 and 966 by Shining World Health Care (Taipei, Taiwan); NE-C21 and NE-C25 by Omron; Model 6610 by Lumiscope; Mist II Model Number 40-270-000 by MABIS; and UM20-1.6 by Hielscher.

7. The system of claim 1 wherein said face mask is a disposable face mask and wherein said face mask is capable of achieving a tight, hermetic fit with a patient's face.

8. The system of claim 7 wherein said face mask is selected from the group consisting of: the Pocket Mask™ by Laerdal Medical Corporation, the self-sealing mask by Vital Signs, and the 1800™, 1812™, 1838™, 1860™, and 8210™ brand masks by the 3M Company.

9. The system of claim 1 wherein said filtration unit employs a filtration device selected from the group consisting of: a micropore filter, sphagnum moss, activated charcoal, an affinity reagent, and an electrical charge-based filter.

10. The system of claim 9 wherein said filtration unit comprises an electrically charged melt-blown microfiber (BMF).

11. The system of claim 9 wherein said filtration unit comprises a spun-bonded nonwoven fibrous media.

12. The system of claim 9 wherein said filtration unit further comprises a fluorochemical additive.

13. The system of claim 9 wherein said filtration unit comprises an active particulate.

14. The system of claim 9 wherein said filtration unit comprises a porous membrane having a pore size of between about 10 nm and about 100 μm.

15. The system of claim 14 wherein said pore size is between about 0.1 μm and about 10 μm.

16. The system of claim 14 wherein said porous membrane is produced from a material selected from the group consisting of a polytetrafluoroethylene, a thermoplastic polymer, and polypropylene.

17. The system of claim 1 wherein said filtration unit further comprises a useful life indicator.

18. The system of claim 17 wherein said useful life indicator employs a signal selected from the group consisting of an optical signal, an auditory signal, and an electrical signal.

19. The system of claim 18 wherein said useful life indicator comprises an agent that reacts with the drug and/or medication.

20. The system of claim 18 wherein said useful life indicator is an active indicator that comprises a sensor and a signaling device.

21. The system of claim 1 wherein said drug and/or medication to be administered is selected from the group consisting of an anesthetic, a sedative, an analgesic, a paralytic, a neuromuscular blocker, a reversal agent, an antihistamine, an anxiolytic, an anticholinergic, and an antihistaminergic.

22. The system of claim 21 wherein said drug and/or medication is ketamine.

23. A system for the non-invasive administration of a drug and/or medication to a patient, said system comprising:
(a) a face mask; and
(b) a nebulizer;
wherein said nebulizer is sealably and insertably connected to said face mask at a first one-way valve;
wherein said first one-way valve prevents the flow of said drug and/or medication from said face mask back into said nebulization unit.

24. A method for the non-invasive administration of a drug and/or medication to a patient, said method comprising the steps of:
(a) attaching to the face of said patient a system of any one of claims 1-22;
(b) adding a unit dose of said drug and/or medication to said nebulizer;
(c) nebulizing said drug and/or medication such that a drug and/or medication containing aerosol is provided to said face mask; and
(d) scavenging exhaled and/or excess drug and/or medication exiting said face mask in a filtration unit.
